(19) United States
(12) Patent Application Publication
(10) Pub. No.: US 2022/0092526 A1
(43) Pub. Date: Mar. 24, 2022

(54) CONTROLLED ORDERING OF SUPPLIES FOR MEDICAL DEVICES AND SYSTEMS

(71) Applicant: Dexcom, Inc., San Diego, CA (US)

(72) Inventors: Brian DALFORNO, Vista, CA (US); Thomas HALL, San Diego, CA (US); Matthew Ryan HUGEL, San Diego, CA (US); Katherine Yerre KOEHLER, Solana Beach, CA (US)

(21) Appl. No.: 17/457,866

(22) Filed: Dec. 6, 2021

Related U.S. Application Data
(63) Continuation of application No. 15/386,934, filed on Dec. 21, 2016.
(60) Provisional application No. 62/271,912, filed on Dec. 28, 2015.

(51) Int. Cl. G06Q 10/08; G16H 40/63; G16H 40/20; G06Q 10/10; G06Q 40/08

(52) U.S. Cl. CPC G06Q 10/087; G16H 40/63; G06Q 40/08; G06Q 10/10; G16H 40/20

(57) ABSTRACT

Disclosed are systems, methods and devices for controlling the ordering of medical device supplies. In some aspects, a method includes authorizing a user to submit an order for supplies associated with a prescribed medical device; receiving information associated with the authorized user including (i) eligible items permitted to be purchased such as the user's prescription for the medical device and/or compatibility of components associated with the medical device, and (ii) pricing of the eligible items based on an insurance plan of the user; determining eligibility of the user to purchase the one or more supplies submitted in the order based on analysis the information; and determining payment information for payers associated with purchase of the determined eligible one or more supplies submitted in the order, in which the payment information for payers include a co-payment for the user and a covered payment for a carrier of the insurance plan.

### CROSS-REFERENCE TO RELATED APPLICATIONS

This application is a continuation of U.S. patent application Ser. No. 15/386,934, filed Dec. 21, 2016, which claims the benefit of U.S. Provisional Application No. 62/271,912, filed Dec. 28, 2015, the entire contents of each of which are incorporated by reference herein.

### TECHNICAL FIELD

The present disclosure generally relates to systems and processes for controlling purchase and distribution of prescribed medical device supplies, such as components and accessories, with one example being those for continuous analyte monitoring.

### BACKGROUND

Diabetes mellitus is a disorder in which the pancreas cannot create sufficient insulin. In a diabetic state, a person suffering from high blood sugar may experience an array of physiological side effects associated with the deterioration of small blood vessels. These side effects may include, for example, kidney failure, skin ulcers, bleeding into the vitreous of the eye, and the like. A hypoglycemic reaction, such as a low blood sugar event, may be induced by an inadvertent overdose of insulin, or after a normal dose of insulin or glucose-lowering agent. In a severe hypoglycemic reaction, there may be a high risk for headache, seizure, loss of consciousness, and coma.

A diabetic person may carry a self-monitoring blood glucose (SMBG) monitor which typically requires the user to prick his or her finger to measure his or her glucose levels. Given the inconvenience associated with traditional finger pricking methods, it is unlikely that a diabetic will take a timely SMBG measurement and, consequently, may be unaware whether his or her blood glucose value is indicative of a dangerous situation.

Consequently, a variety of non-invasive, transdermal (e.g., transcutaneous) and/or implantable electrochemical sensors are being developed for continuously detecting and/or quantifying glucose values. These devices generally transmit raw or minimally processed data for subsequent analysis at a remote device. The remote device may have a display that presents information to a user hosting the sensor. In some systems, a patient may check his or her glucose level on a hand held computing device. There are challenges to presenting this information discreetly and reliably. Similarly, there are challenges in efficiently replacing or replenishing components and accessories of such continuous electrochemical sensor devices, e.g., including continuous glucose monitors (CGM).

### SUMMARY

Methods, systems and apparatuses, including computer program products, are provided for controlled ordering.

In some example embodiments, the present technology includes a method for controlling ordering of supplies for a medical device. The method includes determining, at a server, authorization of a user to submit an order for one or more supplies associated with a medical device prescribed to the user; receiving, at the server, information associated with the authorized user, wherein the information includes (i) eligible items permitted to be purchased by the user based on one or more of (a) the user's prescription for the medical device or (b) compatibility of components associated with the medical device, and (ii) pricing of the eligible items associated with the medical device based on an insurance plan of the user; determining, at the server, eligibility of the user to purchase the one or more supplies submitted in the order based on analysis the information; and determining, at the server, payment information for payers associated with purchase of the determined eligible one or more supplies submitted in the order, in which the payment information for payers include a co-payment for the user and a covered payment for a carrier of the insurance plan.

In some example embodiments, the present technology includes a system for controlling ordering of supplies for a medical device. The system includes a controlled ordering software application operable on a user computing device to (i) receive user input indicative of an order for the one or more supplies associated with a medical device prescribed to a patient user, and (ii) cause transmission of the user input by the user computing device; and one or more secure computers including at least one processor and at least one memory including program code which when executed by the at least one processor causes operations of the one or more secure computers, comprising: receiving the user input, determining authorization of the patient user to submit the order for one or more supplies associated with a medical device, processing ordering information associated with the authorized patient user, wherein the ordering information includes (i) eligible items permitted to be purchased by the patient user based on one or more of (a) the patient user's prescription for the medical device or (b) compatibility of components associated with the medical device, and (ii) pricing of the eligible items associated with the medical device based on an insurance plan of the patient user, determining eligibility of the patient user to purchase the one or more supplies submitted in the order based on the processing of the ordering information, and determining payment information for payers associated with purchase of the determined eligible one or more supplies submitted in the order, wherein the payment information for payers include a co-payment for the patient user and a covered payment for a carrier of the insurance plan.

In some example embodiments, the present technology includes a method for controlling ordering of components and accessories for medical devices, the method including generating a first user interface view to enable presentation and/or selection of at least one receiver for a continuous analyte monitoring system; receiving, at a server, a first selection of the at least one receiver; determining, by the server using compatibility information, at least one transmitter that is compatible to the selected at least one receiver; generating a second user interface view to enable presentation and/or selection of the at least one transmitter; receiving, at the server, a second selection of the at least one transmitter; determining, by the server using compatibility information, at least one sensor that is compatible to the selected at least one transmitter; generating a third user interface view to enable presentation and/or selection of the at least one sensor; receiving, at the server, a third selection of the at least one sensor; and initiating a delivery of the selected receiver, the selected at least one transmitter, and/or the selected at least one sensor.

In some embodiments, one of more variations may be made as well as described in the detailed description below and/or as described in the following features. A determination may be made regarding whether a host-patient using the continuous analyte monitoring system has a single account at the server to enable controlled ordering associated with the continuous analyte monitoring system. A determination may be made regarding whether the host-patient is logged into the single account. A determination may be made regarding whether access, by the host-patient accessing the server, includes a token indicative of having the single account at the server, when the patient is not logged in to the single account. A determination may be made regarding whether an email associated with the host-patient is a unique email in the server, when the access does not include the token. The single account may be created, when patient information obtained from the host-patient accessing the server indicates the host-patient does not have the single account. A first check of a database may be performed by querying existing accounts for the host-patient's last name, date of birth, and at least a portion of a serial number of a receiver, a transmitter, and/or a sensor associated with the host-patient. A second check of a database may be performed by querying the existing accounts for the host-patient's phone number, when the first check fails to find a matching account for the host-patient. An eligibility engine at the server may determine the at least one receiver, the at least one transmitter, and/or the at least one sensor using the compatibility information comprising: previous purchases related to the at least one receiver, the at least one transmitter, and/or the at least one sensor; identity information for the at least one receiver, the at least one transmitter, and/or the at least one sensor; a software version being implemented at the at least one receiver, the at least one transmitter, and/or the at least one sensor; and/or a model of the at least one receiver, the at least one transmitter, and/or the at least one sensor. The compatibility information may map a given receiver, to one or more transmitters, and the compatibility information may also map a given transmitter to one or more receivers. The eligibility engine may access an insurance server including information indicative of whether the host-patient has insurance coverage for purchases associated with the continuous analyte monitoring system. The eligibility engine may query, via a cache, the insurance server. The cache may aggregate queries made to the insurance server. The eligibility engine may further access the insurance server to determine co-payment information for purchases associated with the continuous analyte monitoring system. The eligibility engine may control ordering in a receiver, transmitter, and sensor sequence. The receiving, at the server, the first selection may include receiving the first selection from the host-patient via a remote server or a display device comprising a smartphone and a tablet. The receiving, at the server, the second selection may include receiving the second selection from the host-patient via the remote server or the display device. The receiving, at the server, the third selection may include receiving the third selection from the host-patient via the remote server or the display device. The first user interface view, the second user interface view, and the third user interface view may be presented at the remote server or the display device to enable selection by the host-patient.

It is to be understood that both the foregoing general description and the following detailed description are exemplary and explanatory only and are not restrictive. Further features and/or variations may be provided in addition to those set forth herein. For example, the implementations described herein may be directed to various combinations and subcombinations of the disclosed features and/or combinations and subcombinations of several further features disclosed below in the detailed description.

### BRIEF DESCRIPTION OF THE DRAWINGS

The accompanying drawings, which are incorporated herein and constitute a part of this specification, show certain aspects of the subject matter disclosed herein and, together with the description, help explain some of the principles associated with the subject matter disclosed herein. In the drawings,

FIG. 1 illustrates a continuous analyte sensor system, in accordance with some embodiments;

FIG. 2 illustrates a functional block diagram of a mobile device used with the continuous analyte sensor system, in accordance with some embodiments;

FIG. 3 illustrates an example of a process for ensuring that a patient has only a single account at a server, in accordance with some embodiments;

FIG. 4 illustrates an example of a process for determining whether a patient is authorized to order from the server, in accordance with some embodiments;

FIG. 5 illustrates an example of a process for ensuring compatibility of the components ordered for the continuous analyte sensor system, in accordance with some embodiments;

FIGS. 6A, 6B, and 6C depict examples of user interface views for ordering components for the continuous analyte sensor system, in accordance with some embodiments;

FIGS. 7A, 7B, 7C, and 7D depict additional examples of user interface views for ordering components for the continuous analyte sensor system, in accordance with some embodiments; and

FIGS. 8A, 8B, 8C, and 8D depict examples of user interface views for ordering components for the continuous analyte sensor system, in accordance with some embodiments.

Like labels are used to refer to same or similar items in the drawings.

### DETAILED DESCRIPTION

When a host-patient orders supplies such as components, accessories or other items associated with a medical device, such as a continuous glucose monitoring system, the ordering process can be extremely complex. Specifically, the system components being ordered must be compatible to ensure successful and uninterrupted system operation. For example, if a user orders a new transmitter for the continuous glucose monitoring system and this transmitter is not compatible with a receiver of the continuous glucose monitoring system, then the continuous glucose monitoring system may not operate properly or not at all. In this example, the host-patient may not be able to monitor his/her glucose, which can pose a serious health risk to the host-patient. Moreover, certain insurance providers may place limits on when or how often components can be reordered. Additionally, these providers can each have different pricing schemes.

To illustrate, Jane may have a continuous glucose monitoring system including a receiver for displaying sensor data, a transmitter for providing sensor data to the receiver, and a sensor coupled to the transmitter and affixed to the skin of the host patient. In this example, Jane may want to order a newer version of her current transmitter. However, certain versions of the receiver may not operate with certain versions of the transmitter. Likewise, Jane's insurance (or her prescription) may place limits on when and/or how often Jane can purchase a transmitter.

The present technology provides a controlled ordering system and method for purchasing and/or distribution of medical device supplies that ensures that compatible components, accessories and other items associated with the medical device are efficiently provided to a host-patient. In some embodiments, the controlled ordering systems and methods of the disclosed technology may provide checks of insurance companies to ensure that a host-patient is eligible to purchase a component. For example, a check may include determining whether the host-patient can upgrade to a new component. In some embodiments, the controlled ordering systems and methods of the disclosed technology may provide pricing for the purchase of the supplies based on the host-patient's insurance. In some embodiments, the controlled ordering systems and methods of the disclosed technology may authenticate the host-patient to make sure the ordering is associated with the correct host patient, while maintaining that patient's privacy.

Before providing additional details for the controlled ordering systems and methods noted above, the following provides an example system description in which the controlled ordering may be implemented.

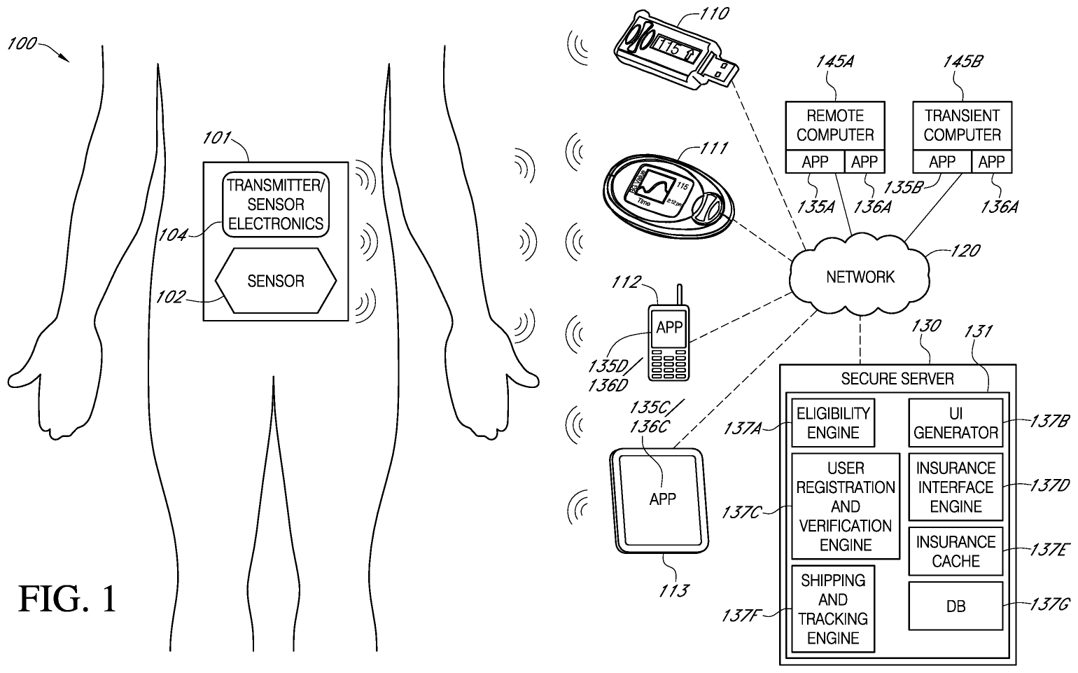

FIG. 1 is a schematic view of a continuous analyte sensor system 100 coupled to a host, such as a patient, and communicating with a number of example devices 110-113, in accordance with some embodiments.

A transcutaneous analyte sensor system 100 comprising an on-skin sensor assembly 101 is fastened to the skin of a host via a disposable housing (not shown). The system includes a transcutaneous analyte sensor 102 and a transmitter/sensor electronics unit 104 for wirelessly transmitting analyte information to a receiver or CGM receivers, such as devices 110-113. In some alternative embodiments, the sensor may be non-invasive.

During use, a sensing portion of the sensor 102 is under the host's skin, and a contact portion of the sensor 102 is electrically connected to the electronics unit 104. The electronics unit 104 engages a housing (not shown), and the sensor extends through the housing. The housing, which maintains the assembly 101 on the skin and provides for electrical connection of the sensor 102 to sensor electronics provided in the electronics unit 104, is attached to an adhesive patch fastened to the skin of the host.

The on-skin sensor assembly 101 may be attached to the host with an applicator (not shown) adapted to provide convenient and secure application. Such an applicator may also be used for attaching the electronics unit 104 to a housing, inserting the sensor 102 through the host's skin, and/or connecting the sensor 102 to the electronics unit 104. Once the electronics unit 104 is engaged with the housing and the sensor 102 has been inserted and is connected to the electronics unit 104, the applicator detaches from the sensor assembly.

The continuous analyte sensor system 100 includes any sensor configuration that provides an output signal indicative of a concentration of an analyte. The output signal, which may be in the form of, for example, sensor data, such as a raw data stream, filtered data, smoothed data, and/or otherwise transformed sensor data, is sent to a receiver, which is described in more detail below. In various embodiments, for example, the analyte sensor system 100 includes a transcutaneous glucose sensor, a subcutaneous glucose sensor, a continuous refillable subcutaneous glucose sensor, or a continuous intravascular glucose sensor, or other type glucose sensor that measures glucose continuously.

The sensor 102 may extend through a housing (not shown), which maintains the sensor on the skin and provides for electrical connection of the sensor-to-sensor electronics, provided in the electronics unit 104. The sensor 102 may be formed from a wire. For example, the sensor can include an elongated conductive body, such as a bare elongated conductive core (e.g., a metal wire) or an elongated conductive core coated with one, two, three, four, five, or more layers of material, each of which may or may not be conductive. The elongated sensor may be long and thin, yet flexible and strong. For example, in some embodiments the smallest dimension of the elongated conductive body is less than about 0.1 inches, 0.075 inches, 0.05 inches, 0.025 inches, 0.01 inches, 0.004 inches, or 0.002 inches. A membrane system may be deposited over at least a portion of electroactive surfaces of the sensor 102 (including a working electrode and optionally a reference electrode) and provides protection of the exposed electrode surface from the biological environment, diffusion resistance (limitation) of the analyte if needed, a catalyst for enabling an enzymatic reaction, limitation or blocking of interferents, and/or hydrophilicity at the electrochemically reactive surfaces of the sensor interface.

The membrane system may include a plurality of domains, for example, an electrode domain, an interference domain, an enzyme domain (for example, including glucose oxidase), and a resistance domain, and can include a high oxygen solubility domain, and/or a bioprotective domain. The membrane system may be deposited on the exposed electroactive surfaces using known thin film techniques (for example, spraying, electro-depositing, dipping, etc.). In some embodiments, one or more domains are deposited by dipping the sensor into a solution and drawing out the sensor at a speed that provides the appropriate domain thickness. However, the membrane system can be disposed over (or deposited on) the electroactive surfaces using any known method.

The electronics unit 104 may be releasably attachable to the sensor 102, which together form the on-skin sensor assembly 101. The electronics unit 104 may include electronic circuitry associated with measuring and processing the continuous analyte sensor data, and is configured to perform algorithms associated with processing and/or calibration of the sensor data. The electronics unit 104 may include hardware, firmware, and/or software that enable measurement of levels of the analyte via a glucose sensor, such as the analyte sensor 102. For example, the electronics unit 104 can include a potentiostat, a power source for providing power to the sensor 102, other components useful for signal processing and data storage, and preferably a telemetry module for one- or two-way data communication between the electronics unit 104 and one or more receivers, repeaters, and/or display devices, such as the devices 110-113. Sensor electronics within the electronics unit 104 can be affixed to a printed circuit board (PCB), etc., and can take a variety of forms. For example, the electronics can take the form of an integrated circuit (IC), such as an application-specific integrated circuit (ASIC), a microcontroller, and/or a processor. The electronics unit 104 may include sensor electronics that are configured to process sensor information, such as storing data, analyzing data streams, calibrating analyte sensor data, estimating analyte values, comparing estimated analyte values with time corresponding measured analyte values, analyzing a variation of estimated analyte values, such as estimated glucose values (EGVs), and/or the like.

The devices 110-113 (also referred to herein more generally as “receivers” or “CGM receivers”) may operate as repeaters, receivers, and/or display devices. In the example of FIG. 1, device 110 comprises a key fob repeater 110, device 111 comprises a dedicated medical device receiver 111, device 112. comprises a smartphone 112 including an application such as a CGM application to provide the receiver disclosed herein, device 113 comprises a portable or tablet computer 113 including an application such as a CGM application to provide the receiver disclosed herein, although other types of devices capable of receiving, repeating, and/or displaying the CGM sensor data provided by electronics unit 104 may be used as well.

Devices 110-113 may be operatively linked (via wireless link(s)) to the electronics unit 104. The repeaters, receivers, and/or display devices 110-113 may receive data from electronics unit 104, which is also referred to as the transmitter and/or sensor electronics body herein. For example, the sensor data can be transmitted from the sensor electronics unit 104 to one or more of the key fob repeater 110, the medical device receiver 111, the smartphone 112, the portable or tablet computer 113, and the like. In some implementations, the repeaters, receivers and/or display devices may also transmit data to the electronics unit 104. In some implementations, the repeaters, receivers, and/or display devices may transmit data to one another or to other servers and/or computers. For example, smartphone 111 may receive CGM data from transmitter 104. Smartphone 111 may display the CGM data as well as related alerts and the like. Smartphone 111 may also provide the CGM data to other devices, such devices 110, 112, 113, as well as one or more other servers, such as secure server 130, via for example network 120.

Data output from the on-skin sensor assembly 101 can be provided via wired and/or wireless, one- or two-way communication between the receiver 110-113 and an external device. The external device can be any device that interfaces or communicates with the receiver. In some embodiments, the external device is a computer, and the receiver is able to download current and/or historical data for retrospective analysis by a physician, for example. In some embodiments, the external device is a modem, and the receiver is able to send alerts, warnings, emergency messages, etc., via telecommunication lines to another party, such as a doctor and/or a family member, at a remote computer 145A-B. In some embodiments, the external device is an insulin pen or insulin pump, and the receiver is able to communicate therapy recommendations, such as an insulin amount and a time to the insulin pen or insulin pump. The external device can include other technology or medical devices, for example pacemakers, implanted analyte sensor patches, other infusion devices, telemetry devices, etc. The receiver may communicate with the external device, and/or any number of additional external devices, via any suitable communication protocol, including radio frequency (RF), Bluetooth, universal serial bus (USB), any of the wireless :local area network (WLAN) communication standards, including the IEEE 802.11, 802.15, 802.20, 802.22 and other 802 communication protocols, ZigBee, wireless (e.g., cellular) telecommunication, paging network communication, magnetic induction, satellite data communication, CPRS, 3G, 4G, 5G, LTE, ANT, and/or a proprietary communication protocol.

Remote computer 145A may be accessed by the host-patient or others (when authorized) to access CGM data and/or CGM reports, and the data and reports may be obtained from the devices 111-113 or secure server 130. For example, remote computer 145A may be utilized by the host-patient, a remote follower (such as a parent or the like assisting in the (racking of the host-patient's CGM data), or other user, e.g., using a mobile computing device such as a smartphone, tablet, and/or wearable device (e.g., smartglasses, smartwatch or the like) executing a software application to provide remote monitoring capabilities of the host-patient's analyte state, e.g., such as by receiving or retrieving permissible data of the host-patient's analyte state and/or receiving notifications associated. with the host-patient's analyte state.

In some embodiments, remote computer 145A may download from a server, such as secure server 130, an application such as CGM application 135A for receiving, transmitting, and/or displaying COM data as well as other types of data.

In some embodiments, the remote computer 145A may include a controlled ordering application 136A, although the controlled ordering application 136A may be included as part of the CGM application 135A as well. The controlled ordering application 136A (as well as controlled ordering applications 1369-D) may be downloaded from a website or server, such as an application store and/or from secure server 130. Alternatively or additionally, controlled ordering application 136A-D may be implemented wholly or in part as a browser that accesses via a website or portal secure server 130 providing one or more of the controlled ordering aspects disclosed herein.

The secure server 130 may have at least one processor and at least one memory storage device, such as a database, that receives, stores, and processes data received from one or more of the key fob repeater 110, the medical device receiver 111, the smartphone 112, the portable tablet computer 113, and other devices. Secure server 130 may include one or more computers in communication with one another, where various modules of the secure server 130 may be wholly or partially stored on one or multiple of the computer(s) of the secure server 130. In some implementations, the secure server 130 includes a network of computers (e.g., servers), operating in the ‘cloud’, to execute the operations of the secure server 130 described herein. In some embodiments of the secure server 130, some modules are stored and operated on one or more secure computers while other modules are stored and/or operated on one or more non-secure computers. In such embodiments, secure server 130 may comprise both secure and non-secure computers to manage and process data both securely and in a manner that does not require certain data to be secured. A remote device may couple to the secure server 130 to access sensor data associated with a given host coupled to the sensor/transmitter. For example, a caregiver, a parent, and/or the like at a computer 145A or 145B may receive, from the secure server or other device, sensor data, reports, associated alerts, and the like to remotely follow a host-patient at receiver 112. The secure server 130 may be secure in the sense that patient data may be secured in order to restrict access to a patient's private data, such as the patient's CGM data, patient identifiable data, therapy recommendations, other device data, and/or the like.

In some embodiments, the secure server 130 may include one or more modules 131 to provide the controlled ordering of GGM related devices, although other types of medical devices may be ordered as well. In some embodiments, the secure server 130 may include an eligibility engine 137A, a user interface generator 137B, a user registration and verification engine 137C, an insurance interface engine 137D, and insurance cache 137E, a shipping and tracking engine 137F, and a database 137G.

In sonic embodiments, the eligibility engine 137A may determine, for each order and for a given patient, what components are eligible to be purchased by the patient. The eligibility engine 137A may, for a given patient having an account at secure server 130, determine the patient's existing glucose monitoring system based on previous purchases or interactions with server 130. For example, the server 130 may include, for a given patient having an account at secure server 130, the identity of the sensor assembly 101 including the type or version of the sensor 102; the identity of the transmitter 104 including the type or version of the transmitter 104; the identity of the receiver 112 including the type or software version of that receiver; and/or the identity of other devices or components which can be used with the continuous glucose monitoring system. Although some of the examples described herein refer to a patient performing the ordering, a patient's representative, such as a caregiver, medical provider, and/or the like, may perform the ordering on behalf of the patient as well.

In some embodiments, the eligibility engine 137A may Also include compatibility rules that indicate which types of receivers, transmitters, and sensors can operate with each other. For example, the compatibility rules may indicate for each type of receiver (for example, by receiver model, receiver version, software version of the receiver software, and/or the like), the types of transmitters (by transmitter model, transmitter version, software version of the transmitter software, and/or the like) that can operate properly with the corresponding receiver. Moreover, the compatibility rules may indicate for each type of transmitter, the types of sensors (by sensor model, sensor version, and/or the like) that can operate properly with the corresponding transmitter. Likewise, the compatibility rules may indicate other types of devices that can operate properly with the patient's current continuous glucose monitoring system.

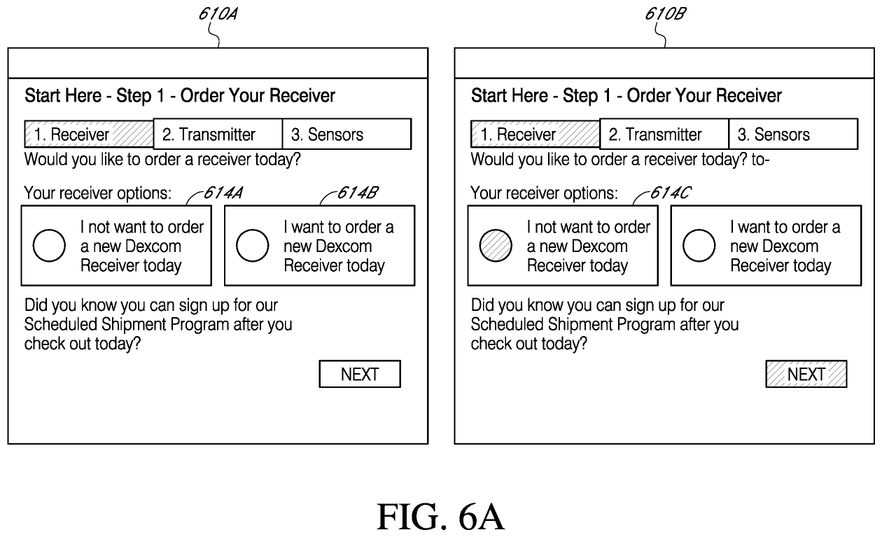

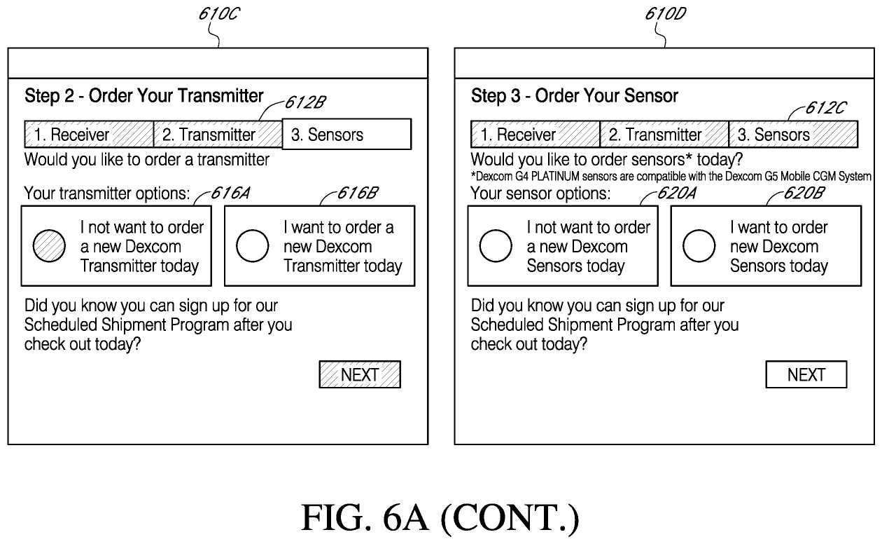

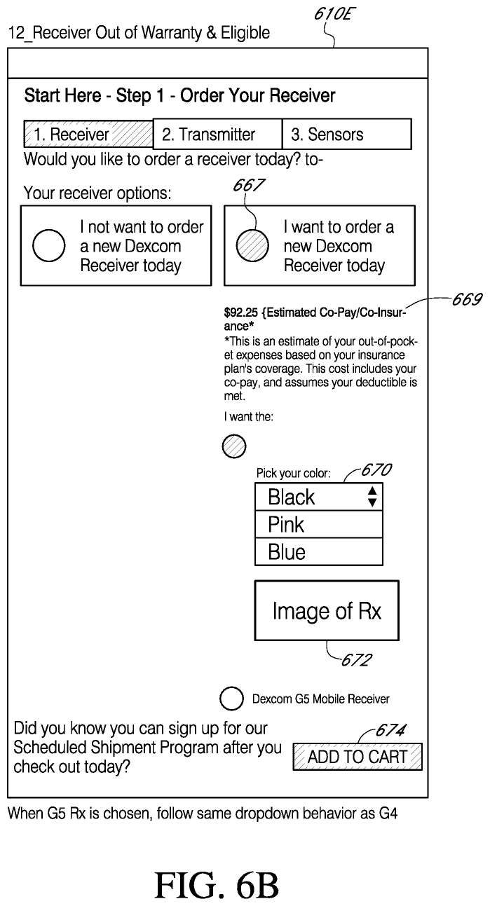

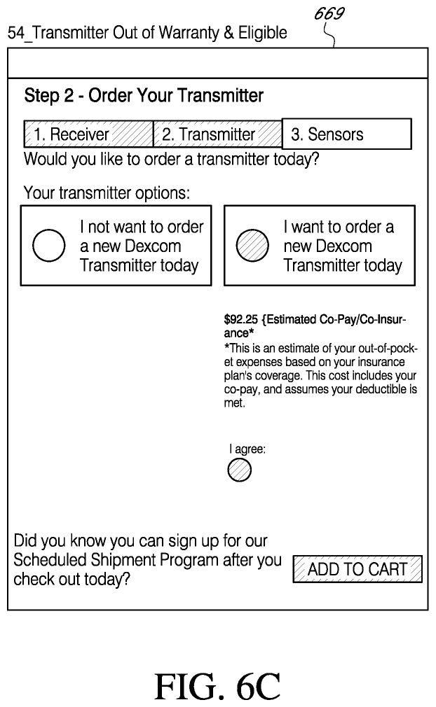

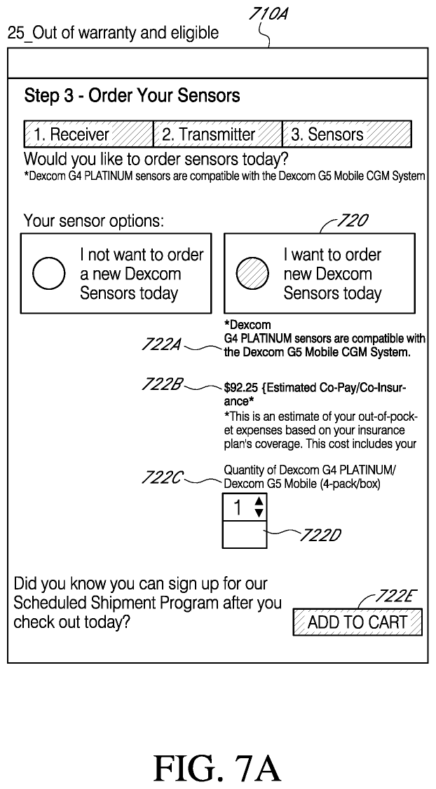

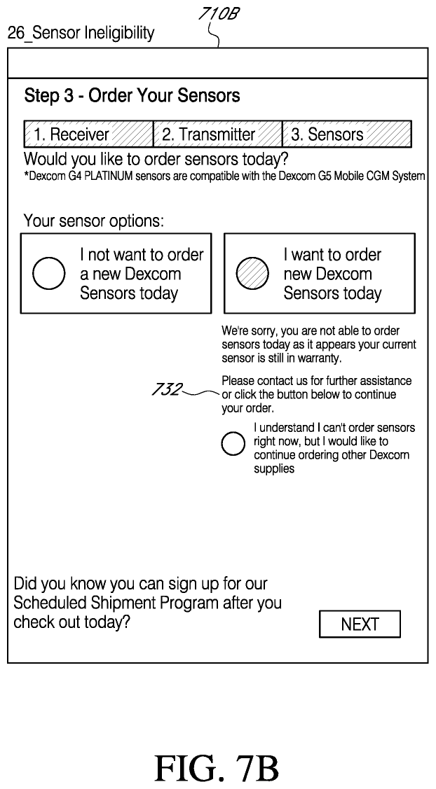

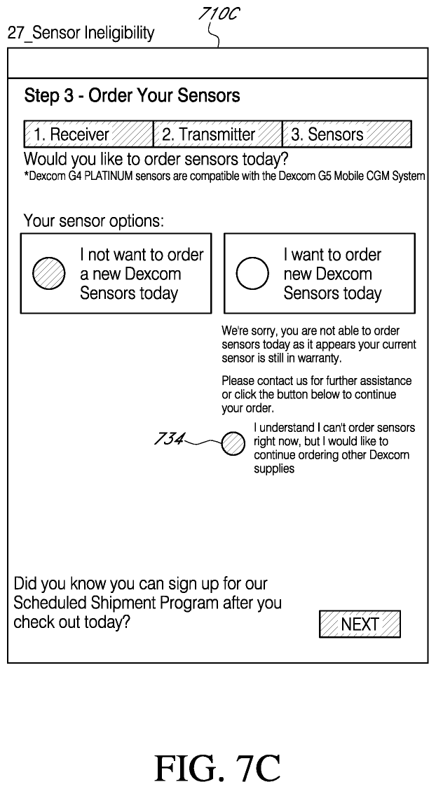

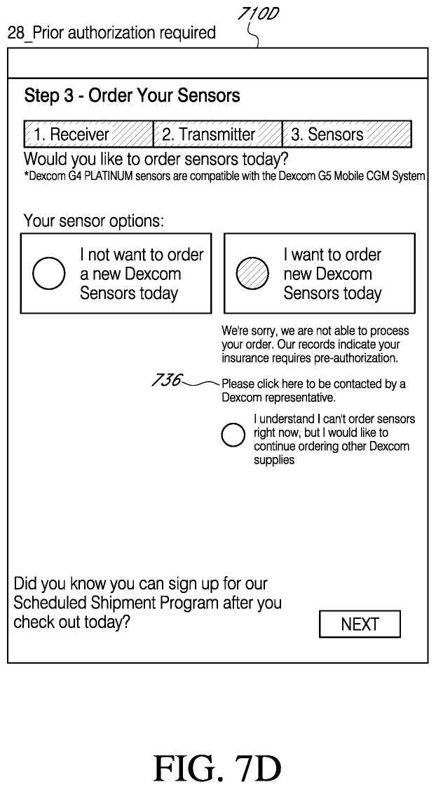

In some embodiments, the user interface (UI) generator 137B may generate user interface views (or provide the information to enable the generation) for presentation via the controlled ordering application 136A-D. Examples of the user interface views are described below with respect to FIGS. 6A-D and FIGS. 7A, 7B, 7C, and 7D.

In some embodiments, the user registration and verification engine 137C may determine whether access to secure server 130 (during the ordering process) is from a new user or from a user that already has an existing account. Because a patient's prescription or insurance provider can limit when or how often a patient can order a receiver, a transmitter, a sensor, or other device, the secure server 130 including the user registration and verification engine 137C may verify that the patient only has a single account. Otherwise, a patient having two accounts for example may purchase twice the quantity of receivers for example than allowed by the patient's prescription or insurance provider.

In some embodiments, the insurance interface engine 137D may provide an interface to servers (also referred to as insurance servers). The insurance servers may provide an indication of whether a patient is covered by insurance, what the patient's coverage is, what the patient's co-pay is, and/or the like. In some embodiments, the insurance cache 137E may be used to store and aggregate accesses to the insurance servers. For example, if four different users are accessing secure server 130 with the controlled ordering applications 136A-D, the insurance cache 137E may store and aggregate the four requests to the insurance servers, rather than make four separate queries to the insurance servers.

In some embodiments, the shipping and tracking engine 137F may be used to track the status of any orders. For example, the shipping and tracking engine 137 may provide to the controlled ordering applications 136A-C estimated shipping dates and/or times, estimated arrival dates and/or times, actual delivery dates and/or times, automatic re-ordering and delivery (for example, re-ordering to resupply the patient automatically), and/or any other delivery information.

In some embodiments, the database 137G may include accounts for one or more patients. The accounts may include information about the patient including the identity of the patient, address, date of birth, phone number, current products being used by the product, past purchases, insurance information, and/or any other information associated with the patient or the patient's devices.

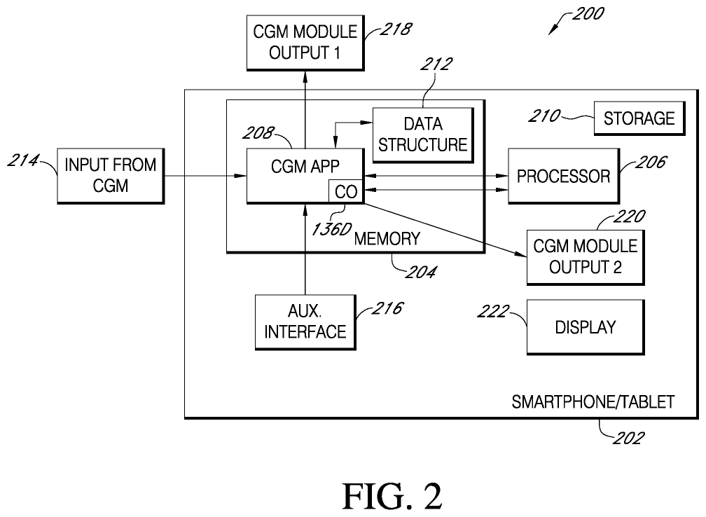

FIG. 2 is a functional block diagram of an embodiment of a system 200 for leveraging mobile device features in continuous analyte monitoring (e.g., such as in a continuous glucose monitoring system), according to some embodiments.

The system 200 may comprise a mobile device, which may be any type of computing device capable of receiving one or more inputs and producing an output. Examples of the mobile device include a smartphone 112, a tablet 113, a laptop, a portable or wearable computing device (e.g., a smartwatch, smartglasses, etc.), and/or the like. The mobile device 202 may include at least one memory 204 and at least one processor 206. The memory 204 may provide the processor 206 access to data and program information that is stored in the memory 204 at execution time. Typically, the memory 204 may include random access memory (RAM) circuits, read-only memory (ROM), flash memory, etc., or a combination thereof. The processor 206 may be, or may include, one or more programmable general-purpose or special-purpose microprocessors 206, digital signal processors 206 (DSPs), programmable controllers, application specific integrated circuits (ASICs), programmable logic devices (PLDs), etc., or a combination of such hardware-based devices.

Processor 206 may execute a continuous glucose monitoring (CGM) application 208 out of the memory 204. The CGM application may, as noted, comprise a browser configured to access via network 120 (for example, the Internet) the secure server 130. In some embodiments, the CGM application is configured to analyze CGM data provided by a transmitter 104 as well as other data provided by other devices. As used herein, the phrase continuous glucose monitoring CGM application should be construed broadly to include not just the application itself, but also integration with sensor 102, other diabetes management devices, including insulin delivery therapies such as insulin pumps, insulin pens, or other drugs useful for the treatment of diabetes. In other words, the CGM application may perform other functions besides monitoring glucose (which may include blood glucose and/or interstitial glucose measurements). It could, for example, determine that a user's glucose level is high, and then transmit a signal to the user's insulin pump to administer a quantity of insulin to bring the user's glucose level down. It could, for example, receive data from another health monitor, medical device, or mobile application of the user that provides contextual data affiliated with the user's glucose state and monitoring, and then process such data with CGM data to inform the user (e.g., via display 222) and/or cause an action in the other device or application. A software and/or firmware component of the CGM application 208 may be stored in storage 210 available to the mobile device 202, and loaded into the memory 204 at execution time. The storage 210 may be any non-transitory computer readable media. including, but not limited to, a hard disk, EEPROM (electrically erasable programmable read only memory), a memory stick, or any other storage device type. The memory 204 may contain one or more data structures 212 that the CGM application 208 can access during execution. The CGM application 208 may be embodied as downloadable software that a user may download from a remote server, such as server 130, through a wired or wireless connection. For example, the user may access the server using an application already installed on the user's mobile device. The user may then download and install the CGM application 208 with the aid of the application. The user may then configure the CGM application 208. For example, the configuration may include setting the user's personal preferences and/or settings, such as contacts, events, modes, profiles, and/or the like. The configuration may be done manually, such as by selecting various options from menus, or automatically.

In some embodiments, processor 206 may execute a controlled ordering application, such as controlled ordering application 136D (labeled “CO”) out of memory 204. As noted above, the controlled ordering application 136D may be implemented wholly or in part as a browser that accesses (via a website or portal, for example) secure server 130 to provide the controlled ordering disclosed herein. A software and/or firmware component of the controlled ordering application 136D may be stored in storage 210 available to the mobile device 202, and loaded into the memory 204 at execution time. The storage 210 may be any non-transitory computer readable media including, but not limited to, a hard disk, EEPROM (electrically erasable programmable read only memory), a memory stick, or any other storage device type. The memory 204 may contain one or more data structures 212 that the controlled ordering application 136D can access during execution. The controlled ordering application 136D may be embodied as downloadable software that a user may download from a remote server, such as server 130, through a wired or wireless data connection, although the controlled ordering application 136D may be downloaded from other servers or websites and/or be provided in other ways as well.

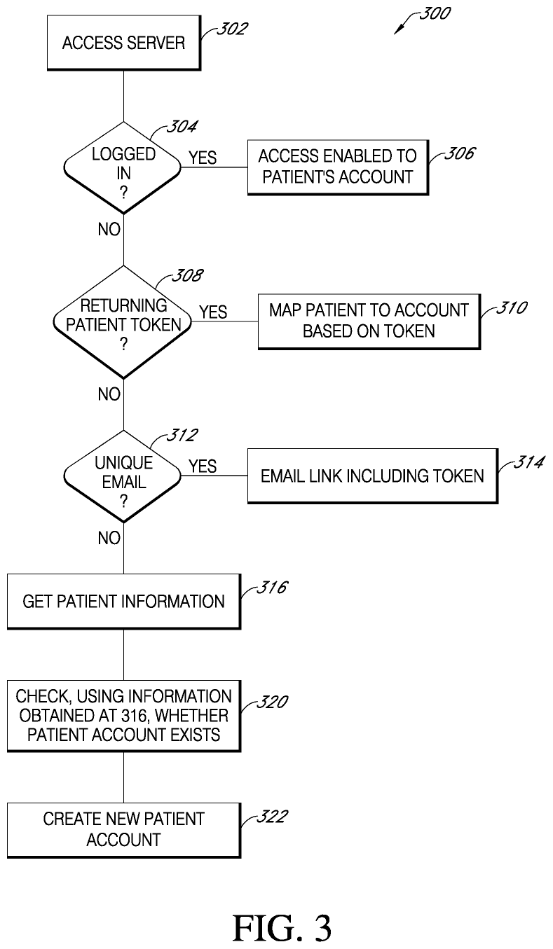

In some embodiments, the controlled order application 136A-D may provide one or more of the aspects described with respect to the processes described below with respect to FIGS. 3, 4, and 5.

FIG. 3 illustrates a process 300 flow illustrating the authentication and registration via the controlled ordering disclosed herein. The description of FIG. 3 also refers to FIG. 1.

Process 300 may be used to ensure that a patient using the controlled ordering application 136A-D is associated with the proper account and to ensure the server 130 does not create two accounts at database 137G for the same patient. Having two accounts for the same patient may lead to difficulties related to ordering products not covered by the patient's prescription or insurance policy. For example, a patient's prescription or insurance policy may allow a certain quantity of sensors per year. If that patient has two accounts, the patient may be improperly allowed to order twice as much as authorized by the patient's prescription or insurance policy.

At 302, a patient seeking to order one or more components may access a computing device, such as smartphone 112 including the controlled ordering application 136D or remote computer 145A including controlled ordering application 136A, to access secure server 130 to place an order.

At 304, the server 130 including user registration and verification engine 137C (also referred to as verifier 137C) may determine whether the patient accessing the server is logged into the patient's account at the server 130. If so, the patient is an authenticated and registered patient with an account at the database 137G. As such, the server 130 may be allowed to access patient account information at the database 137G (yes at 304 and 306). This patient account information may include the identity of the patient (for example, the patient's first and last name), patient's data of birth, patient's home address, patient's phone number, the serial number and/or identities of the patient's current devices (for example, serial number of the patient's receiver, transmitter, sensor, and/or the like), past order information (for example, the identity of past devices purchased by the patient), the patient's insurance provider or policy number, and/or any other information.

At 308, the server 130 including verifier 137C may check to see if the patient is a returning patient based on metadata associated with the access. For example, the smartphone 112 including the controlled ordering application 136D may browse to the server 130. When this occurs, there may be a token in the URL, and this token may be used to uniquely map the patient to the patient's account at server 130 (yes at 308 and 310).

At 312, the server 130 including verifier 137C may check to see if the patient's email is uniquely associated with a single account in the database 137C. If so, the server may send an email link to the patient, and this link may include an indication, such as a token, that maps uniquely to patient's account at the server 130 (yes at 312 and 314). The patient may select the link that navigates the patient's controlled ordering application to a login screen for the patient.

The server 130 including verifier 137C may, at 316, gather information about the patient to determine whether the patient can be uniquely associated with a single account in the database 137C. For example, the verifier 137C may obtain directly from the patient via a user interface view (where the information can be input by the patient) the patient's last name, date of birth, serial number of a receiver (and/or transmitter and/or sensor), patient's phone number, address, and/or the like.

At 320, server 130 including verifier 137C may then check by querying database 137G. The query may include one or more of the information about the patient obtained at 316. The query of the database may check whether the patient has an existing account in database 137G. In some embodiments, verifier 137C may first check the database 137G by querying the existing accounts for any one or more of the patient's last name, date of birth, social security number (or portion thereof), or other patient identification information, and/or at least a portion of a serial number of a device, such as the receiver, the transmitter, and/or the sensor. If that information matches an existing account at database 137G, the patient access is linked to an account by sending an email including account information, such as user name to enable a login to the account. If that information does not match an existing account at database 137G, the verifier 137C may perform a second check, at 320, by for example sending a query using the patient's phone number. If an account is not found in the database 137G with a matching phone number, the patient is classified as a new patient that is not already registered at server 130 and/or database 137G, in which case the server 130 may, at 322 create a new account for the patient to enable patient ordering.

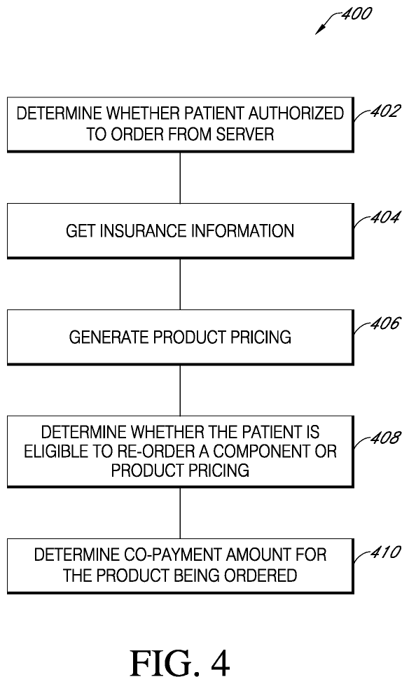

FIG. 4 depicts an example process 400 for determining whether a patient is eligible to receive, e.g., via ordering, one or more components of a prescribed medical device, such as a receiver, a transmitter, a sensor, and/or other device component in a controlled way in accordance with some embodiments. The description of FIG. 4 also refers to features described in FIG. 1.

As noted, when a patient wants to order one or more components, the patient may access a computing device, such as smartphone 112 including the controlled ordering application 136D. For example, the controlled ordering application 136D and server 130 may enable ordering in a controlled way that takes into account the patient's insurance coverage, the patient's prescription, the patient's current system configuration including compatibility among components/devices, upgrade possibilities for the patient's current components/devices, past orders, and/or the like.

At 402, the server 130 including the eligibility engine 137A may determine whether the patient is authorized to receive and/or order component or devices via server 130. For example, the server 130 may only be able to provide components to patients living in a certain jurisdiction or country, such as the US. In this example, when a patient accesses server 130 and logs in to their account, the eligibility engine 137A may determine from information provided by the patient, the address or location of the patient. If the patient is based in the US, the eligibility engine 137A may allow the patient to proceed with the ordering. If the patient is not in the US, the eligibility engine 137A may deny access by sending, for example, a message indicating error or access denied.

At 404, the server 130 including the eligibility engine 137A may access the patient's insurance information as well as other information. For a patient logged into server 130 via the controlled ordering application 136D for example, the eligibility engine 137A may access the patient's account information stored at database 137G. This account information may, as noted, include insurance information, such as the identity of the insurance provider, the patient's policy number, and/or other information about the policy (for example, co-payments, how often and/or when certain components can be ordered, etc.).

At 406, the server 130 including the eligibility engine 137A may generate pricing and available products based on the insurance information obtained at 404. For example, the eligibility engine 137A may query via the insurance interface engine 137D whether the patient's policy is active and thus in effect. The insurance interface engine 137D may also obtain and/or store metadata about the insurance policy including coverage limits indicating how much the insurance company will pay for components or products, how often (or when) the insurance company will pay for components or products (for example, can a new product be purchased annually), the amount of the patient's co-payment, and/or the like.

At 408, the server 130 including the eligibility engine 137A may determine whether the patient is eligible to order or re-order a component or product. For example, the eligibility engine 137A may have a rule that allows a patient to re-order a new receiver if the patient's policy is active policy and the patient has not purchased a receiver in the last two years as specified by the patient's insurance policy information, although other rules may be implemented as well. The rules can be implemented as a table listing each device including model, serial number, and/or software version. For each device, the table may map to one or more compatible devices. In some implementations of the process 400, the process 400 concludes upon determination of whether the patient is eligible to order the component or the product.

Yet in some implementations, the process 400 includes the server 130 (e.g., via the eligibility engine 137A) determining, at 410, payment information for the patient user's payer associated with purchase of the eligible component or product submitted in the order. For example, the payment information for payers can include a co-payment for the patient user and/or a covered payment for a carrier of the patient user's insurance plan. In some implementations, the server 130 including the eligibility engine 137A can obtain the co-payment amount for the product(s) being ordered to enable presentation of the co-payment amount via a user interface view at the controlled ordering application.

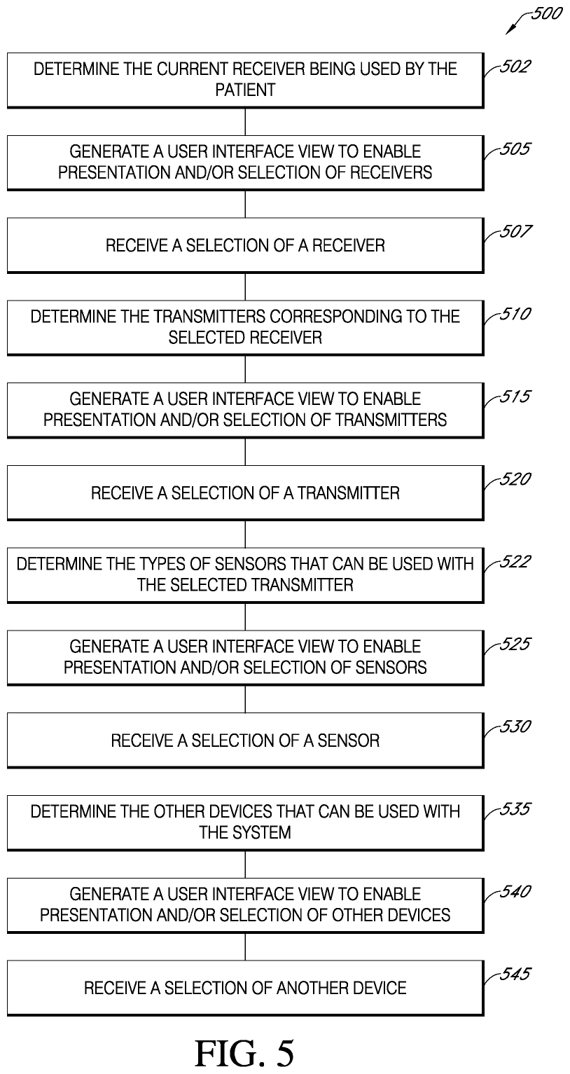

FIG. 5 depicts an example process 500 for determining eligible configurations of a medical device, such as continuous glucose monitoring system including a receiver, a transmitter, and a sensor, in accordance with some embodiments. The description of FIG. 5 also refers to features described in FIG. 1.

In some embodiments, the eligibility engine 137A may control the ordering so that certain components are ordered in a certain sequence. For example, if a receiver is being ordered, it may need to be ordered before a transmitter, which may be followed by the sensor ordering. This sequence is generally in the opposite direction of the sensor data flow (which may be from sensor to transmitter and then to receiver). This sequence may simplify the rules related to identifying compatible devices.

At 502, the server 130 may determine the current receiver being used by the patient. For example, the eligibility engine 137A may access database 137G to determine from the patient's account the current receiver being used by the patient. If that information is not present at the database 137G, the eligibility engine 137A may call the UI generator 137B to generate a UI view for presentation to the patient, and the presented UI view may query the patient to provide the current receiver model or serial number. The UI view may be generated by the UI view generator 137B as markup or other script or code, and sent to a display device for presentation at a display (although the display device may generate at least a portion of the UI view as well).

At 505, the server 130 may generate a user interface view to enable presentation and/or selection of receivers. For example, given the current receiver being used by the patient, the eligibility engine 137A may generate a list of possible receivers that the patient can select from. The list of possible receivers may include the current receiver model being used (for example, if the patient simply wants a new receiver) or another model of the receiver, such as a more current model. Moreover, the eligibility engine 137A may take into account the patient's insurance information indicating whether the patient is eligible to purchase another receiver (for example, based on insurance information indicative of when or how often given the patient's last purchase of a receiver). In some instances, the eligibility engine 137A may take into account the warranty of the patient's current receiver. For example, if the patient's account indicates the patient's receiver was purchased 4 months ago, the eligibility engine 137A may indicate to the patient that the patient is not allowed to purchase another receiver since the receiver is still within the 6-month warranty. In some embodiments, the eligibility engine 137A may generate a list of one or more eligible receivers, and provide that list to UI generator 137B, which generates a UI view including the list of receivers for presentation to the patient. That generated view may then be provided to a display device such as smartphone 112 including the controlled ordering application 136D for presentation at a display.

At 507, the server 130 may receive a selection of a receiver. For example, the patient's smartphone 112 including controlled ordering application 136D may present the UI view including the list of receivers generated at 505. For example, the patient may want to upgrade to a new receiver. A user may then select one of the receivers being displayed. This selection may then trigger a message including the selected receiver to be sent to the secure server 130.

At 510, the server 130 may determine the transmitters corresponding to the selected receiver. For example, the secure server 130 may receive, at 507, a selection of a certain type of receiver. Based on this list, the secure server 130 may access a set of rules that map the selected receiver to one or more compatible transmitters. Moreover, the eligibility engine 137A may take into account the patient's insurance information indicating whether the patient is eligible to purchase another transmitter.

At 515, the server 130 may generate a user interface view to enable presentation and/or selection of transmitters. For example, the eligibility engine 137A may generate a list of one or more eligible transmitters, and provide that list to UI generator 137B, which generates a UI view including the list of transmitters for presentation to the patient.

At 520, the server 130 may receive a selection of a transmitter. For example, the patient's smartphone 112 including controlled ordering application 136D may present the UI view including the list of transmitters generated at 515. A user may then select one of the transmitters being displayed. This selection may then trigger a message including the selected transmitter to be sent to the secure server 130.

At 522, the server 130 may determine the types of sensors that can be used with the selected transmitter. For example, the secure server 130 may, given the transmitter selection information obtained at 520, access a set of rules that map the selected transmitter to one or more compatible sensors. Moreover, the eligibility engine 137A may take into account the patient's insurance information indicating whether the patient is eligible to purchase another sensor.

At 525, the server 130 may generate a user interface view to enable presentation and/or selection of sensors. For example, the eligibility engine 137A may generate a list of one or more eligible sensors, and provide that list to UI generator 137B, which generates a UI view including the list of sensors for presentation to the patient.

At 530, the server 130 may receive a selection of a sensor. For example, the patient's smartphone 112 including controlled ordering application 136D may present the UI view including the list of sensors generated at 525. A user may then select one of the sensors being displayed. This selection may then trigger a message including the selected sensor to be sent to the secure server 130.

In some implementations of the process 500, at 535, the server 130 may determine other devices that can be used with the continuous glucose monitoring system. For example, the secure server 130 may access a set of rules that map the patient's glucose monitoring system to one or more other devices that can be used in conjunction with the continuous glucose monitoring system. For example, the other device may include heart rate monitors, glucose pens, insulin pump, and/or the like. Moreover, the eligibility engine 137A may take into account the patient's insurance information indicating whether the patient is eligible to purchase or order other devices. At 540, the server 130 may generate a user interface view to enable presentation and/or selection of the other devices. At 545, the server 130 may receive a selection of the other devices. At this stage, the patient has one or more items that can be ordered, purchased and shipped to the patient. In some embodiments, the patient may select when and how often the order should be re-ordered, re-purchased, and shipped to the patient. For example, the patient may request that a certain quantity of sensors be shipped and resent to the patient. In this example, one or more aspects of process 500 may be re-executed to confirm that the patient is still eligible to receive the sensors.

In some embodiments of a process for determining eligible configurations of a medical device, the eligibility engine 137A may control the ordering so that multiple or all of the eligible components are presented to the user simultaneously and determine eligibility of particular components of the medical device in real-time as the user selects presented components. In such embodiments, the server 130 may generate a user interface (e.g., a framework of what to display and/or restrict from display) to enable presentation and/or selection of the components or products for the user to order on a singular display. In the example of the medical device being a continuous glucose monitor, the server 130 can generate a user interface of one or more eligible receiver components, one or more eligible transmitter components, and one or more sensor components associated with the continuous glucose monitor, or combinations thereof, in which the user interface includes options for the user to select only eligible components and configurations of such components for the continuous glucose monitor. For example, generation of the user interface, e.g., prior to its presentation on the user device 110-113, includes determining that the transmitter component(s) are compatible to the receiver and/or software operating thereon, and that the sensor component(s) are compatible to the transmitter component(s). For example, the compatibility of the components associated with the continuous glucose monitor among the eligible items for purchase can be based a model of at least some of the components, an identity of at least some of the components, a previous purchase of at least some of the components, and/or a software version of at least some of the components. The determination of eligibility of components capable of being ordered, and thereby included in the generated user interface, can also include analysis of the user's prescription for the continuous glucose monitor, including pricing data set by the user's insurance plan of the eligible items associated with the continuous glucose monitor.

FIG. 6A depicts examples of user interface views 610A-D that may be presented at a display of for example smartphone 112 including controlled ordering application 135D.

User interface view 610A includes a sequence bar 612A. The sequence bar 612A graphically depicts the progression in ordering sequence of ordering a receiver, a transmitter, and/or a sensor. In the example view, the sequence bar 612A graphically shows the receiver ordering stage. User interface view 610A also includes a user interface element 614A where the user indicates that a receiver is not being ordered, and includes a user interface element 614B where the user indicates that a receiver is being ordered. User interface view 610B shows a selection 614C of not wanting to order a receiver, which triggers generation and presentation of user interface element 610C.

User interface 610C shows a sequence bar 612B at the transmitter ordering stage. User interface view 610C also includes a user interface element 616A where the user indicates that a transmitter is not being ordered, and includes a user interface element 616B where the user indicates that a transmitter is being ordered. In this example, the user interface element 616A is shown as selected, which triggers generation and presentation of user interface element 610D.

User interface 610D shows a sequence bar 612C at the sensor ordering stage. User interface view 610D further includes a user interface element 620A where the user indicates that a sensor is not being ordered, and includes a user interface element 620N where the user indicates that a sensor is being ordered.

FIG. 6B depicts examples of user interface views 610E that may be presented at a display of for example smartphone 112 including controlled ordering application 136D. In this example view, the patient has selected ordering a receiver 667, which triggers the patient's co-pay 669. The patient can also select a color or other features of the selected receiver, which in this example corresponds to the color 670 of the receiver. The view 610E may also include an image of the prescription (which in this example was previously uploaded to the database 137G and associated with the patient's account), and a cart icon 674 that triggers generation of a shopping cart user interface view where the final order can be viewed and executed.

FIG. 6C depicts examples of user interface view 669. User interface view 669 is similar in some respect to user interface view 610E, but user interface view 669 shows the transmitter ordering view.

FIGS. 7A, 7B, 7C, and 7D depict examples of user interface views 710A-D that may be presented at a display of for example smartphone 112 including controlled ordering application 135D.

User interface view 710A includes a sequence bar 712A. The sequence bar 712A graphically indicates the sensor ordering stage. The user interface view 710A also includes a selection 720A for wanting to order a sensor. When selection 720A is made, one or more sensors may be presented at 722A along with payment information such as a co-pay amount at 722B and a quantity of sensors to be ordered at 722C. The one or more sensors presented and the payment (or co-payment) amount may, as noted, be determined by the eligibility engine 137A. If the eligibility engine 137A determines that the patient is not eligible, user interface views 710B -D may be generated. User interface view 710B indicates 732 that the patient cannot order because the sensor is still under warranty (so re-ordering and purchase is unnecessary). User interface view 710C includes a patient acknowledgement element 734 so that the patient can acknowledge not being able to order because the sensor is still under warranty 734. User interface view 710D indicates 736 that the patient cannot order because the patient's insurance provide will not allow the purchase (for example, pre-authorization to purchase may be required by the insurance provider), although the insurance provider may not authorize the purchase of the sensor for other reasons as well.

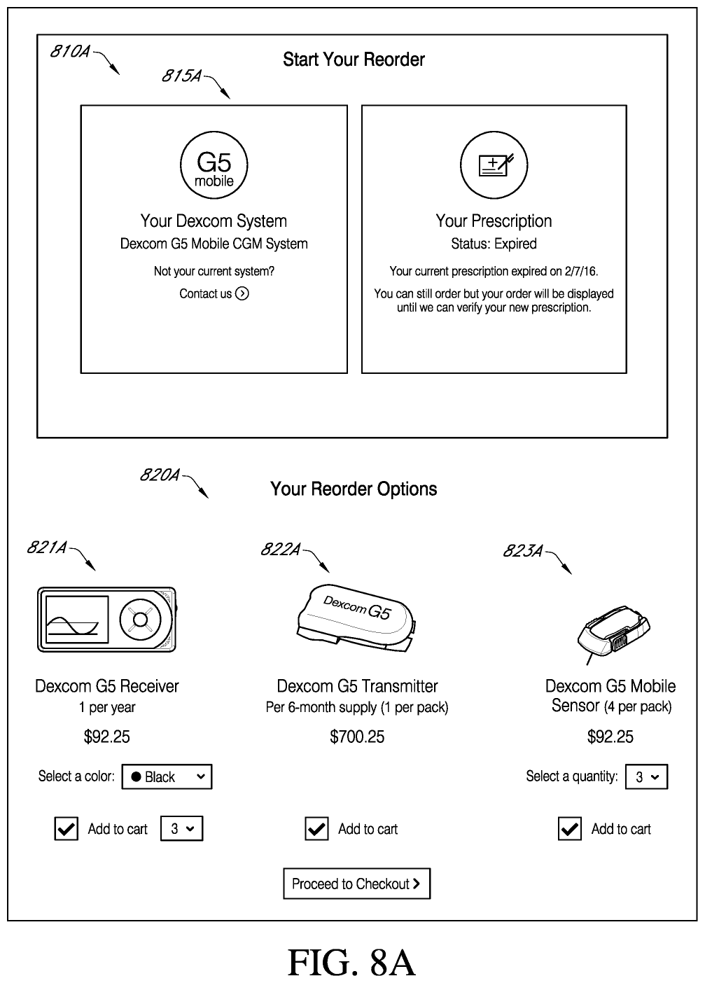

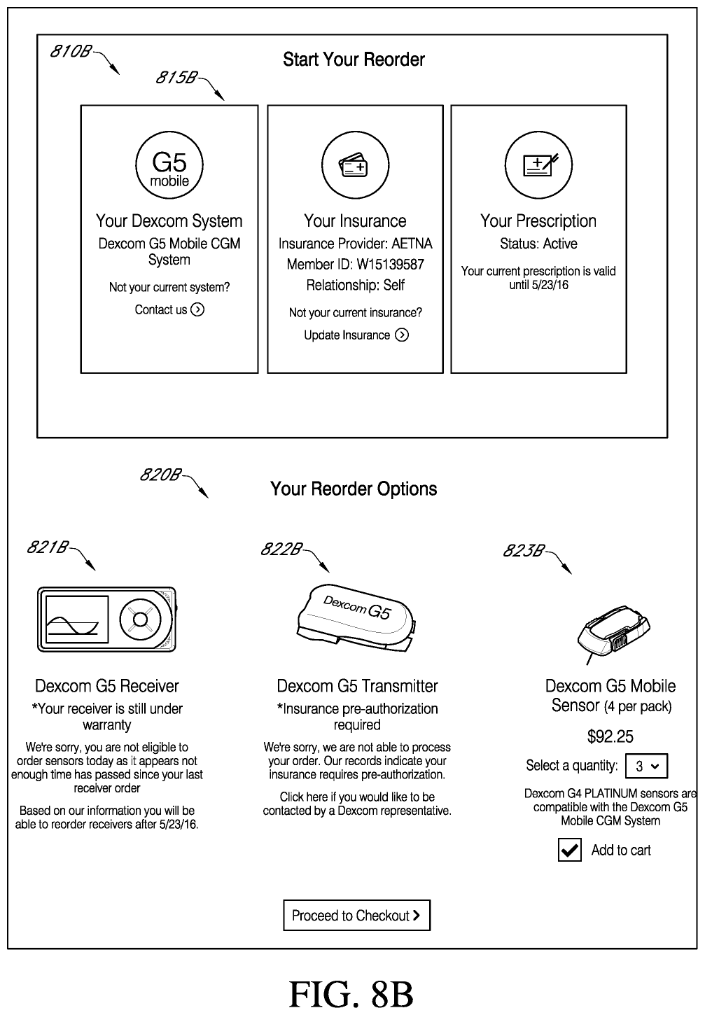

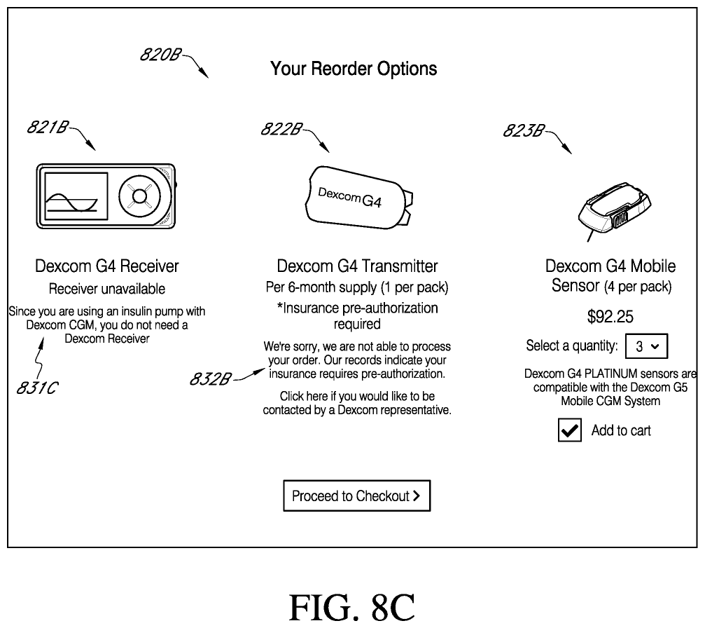

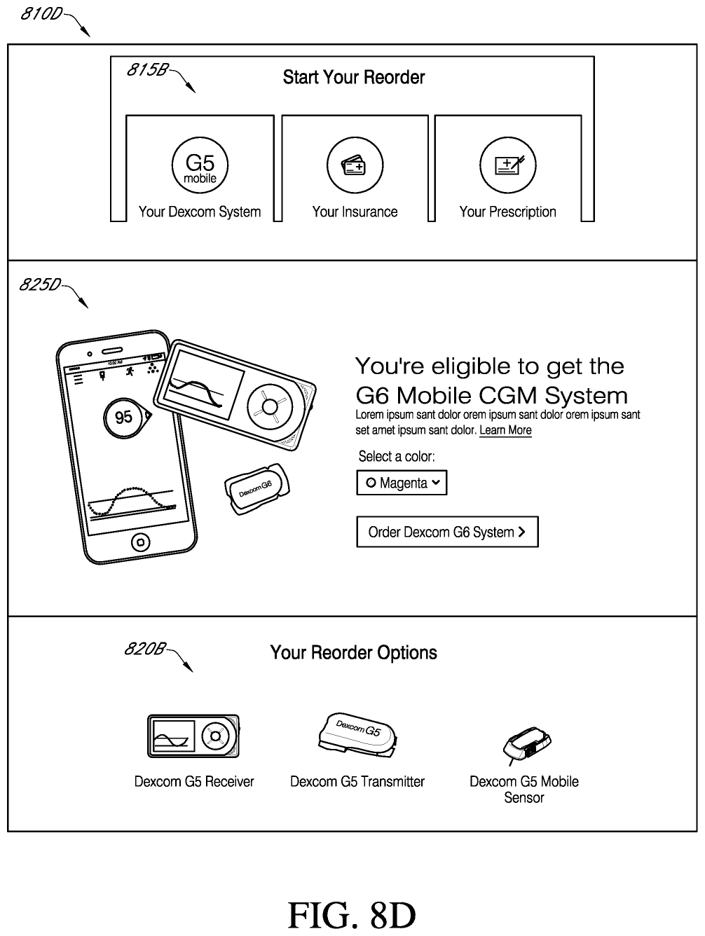

FIGS. 8A-8D depict examples of user interface views 810A, 810B and 810D that may be presented at a display of for example smartphone 112 including controlled ordering application 135D. The example user interface views 810A, 810B and 810D depict user interfaces that simultaneously display components of a medical device, e.g., a continuous glucose monitoring system, determined eligibility for purchase in real-time as the user selects presented components. In some views, the example user interface views 810A, 810B and 810D may show additional information, e.g., such as the user's insurance information, the status of the user's prescription for the medical device and/or particular components, and/or possible upgrade status.

FIG. 8A shows an example of user interface view 810A for re-ordering components of a medical device and displaying the user's current medical device status and prescription status. User interface view 810A includes an information pane 815A that displays the user's system information (e.g., model, series, identify, etc. of the user's continuous glucose monitor) and the status of the user's prescription for the continuous glucose monitor. In this example, the user's prescription status is shown expired. User interface view 810A includes a selection pane 820A of re-order options for a user who wants to re-order a receiver, a transmitter, and/or a sensor for the user's continuous glucose monitor. When selection 821A is made, one or more eligible receivers may be presented along with pricing or payment information and/or other options, e.g., such as receiver device configurations (e.g., color). When selection 822A is made, one or more eligible transmitters may be presented along with pricing or payment information and/or other options. When selection 823A is made, one or more eligible sensors may be presented along with pricing or payment information and/or other options. In some implementations of the selection pane 820A, the eligible quantity available to be ordered during the ordering session can be displayed for each or any of the components.

FIG. 8B shows an example of user interface view 810B for re-ordering components of a medical device and displaying the user's current medical device status, insurance information, and prescription status. User interface view 810B includes an information pane 815B that displays the user's system information (e.g., model, series, identify, etc. of the user's continuous glucose monitor), the user's insurance information (e.g., insurance company name, member id, and/or insured customer, etc.), and the status of the user's prescription for the continuous glucose monitor. In this example, the user's prescription status is shown as active. User interface view 810B includes a selection pane 820B of re-order options for a user who wants to re-order a receiver, a transmitter, and/or a sensor for the user's continuous glucose monitor. In this example user interface view, the selection 821B shows that no receiver components are eligible for purchase by the user during the ordering session, e.g., which can be accompanied by an explanation. In this example, text 831B describes that not enough time has elapsed since the last purchase of the receiver, which is prohibited for purchase under the user's insurance plan. The selection 822B shows that no transmitter components are eligible for purchase by the user during the ordering session, e.g., which can be accompanied by an explanation. In this example, text 832B describes that the user's insurance plan requires a pre-authorization. The selection 823B shows that one or more eligible sensor components are eligible, which also presents pricing or payment information and/or other options. In some implementations of the selection pane 820B, the eligible quantity available to be ordered during the ordering session can be displayed for each or any of the components.

FIG. 8C shows the selection pane 820B of the example user interface view 810B including an alternative explanation why no receiver components are eligible for purchase by the user during the ordering session. The example explanation is depicted by text 831C, which describes that since the user is using an insulin pump associated with continuous glucose monitor, the dedicated receiver is ineligible for purchase.

FIG. 8D shows an example of user interface view 810D for re-ordering components of a medical device and displaying information pertaining to the ordering process. User interface view 810D includes the information pane 815B (shown in part in the drawing of FIG. 8D), the selection pane 820B (shown in part in the drawing of FIG. 8D), and an upgrade pane 825D, e.g., shown between the information pane 815B and the selection pane 820B. The upgrade pane 825D depicts eligible options for ordering that can replace and/or augment at least some of the user's existing components of the medical device. In this example, the upgrade pane 825D provides an option to order an upgraded transmitter component and associated receiver for the user's continuous glucose monitor.

### EXAMPLES

In some embodiments of the present technology (example 1), a method includes generating a first user interface view to enable presentation and/or selection of at least one receiver for a continuous analyte monitoring system; receiving, at a server, a first selection of the at least one receiver; determining, by the server using compatibility information, at least one transmitter that is compatible to the selected at least one receiver; generating a second user interface view to enable presentation and/or selection of the at least one transmitter; receiving, at the server, a second selection of the at least one transmitter; determining, by the server using compatibility information, at least one sensor that is compatible to the selected at least one transmitter; generating a third user interface view to enable presentation and/or selection of the at least one sensor; receiving, at the server, a third selection of the at least one sensor; and initiating a delivery of the selected receiver, the selected at least one transmitter, and/or the selected at least one sensor.

Example 2 includes the method of example 1, further including determining whether a host-patient using the continuous analyte monitoring system has a single account at the server to enable controlled ordering associated with the continuous analyte monitoring system.

Example 3 includes the method of example 2, further including determining whether the host-patient is logged into the single account.

Example 4 includes the method of example 3 further including determining whether access, by the host-patient accessing the server, includes a token indicative of having the single account at the server, when the patient is not logged in to the single account.

Example 5 includes the method of example 4, further including determining whether an email associated with the host-patient is a unique email at the server, when the access does not include the token.

Example 6 includes the method of example 5, further including creating the single account, when patient information obtained from the host-patient accessing the server indicates the host-patient does not have the single account.

Example 7 includes the method of example 6, further including performing a first check of a database by querying existing accounts for the host-patient's last name, date of birth, and at least a portion of a serial number of a receiver, a transmitter, and/or a sensor associated with the host-patient; and performing a second check of the database by querying the existing accounts for the host-patient's phone number, when the first check fails to find a matching account for the host-patient.

Example 8 includes the method of example 1, in which an eligibility engine at the server determines the at least one receiver, the at least one transmitter, and/or the at least one sensor using the compatibility information comprising: previous purchases related to the at least one receiver, the at least one transmitter, and/or the at least one sensor; identity information for the at least one receiver, the at least one transmitter, and/or the at least one sensor; a software version being implemented at the at least one receiver, the at least one transmitter, and/or the at least one sensor; and/or a model of the at least one receiver, the at least one transmitter, and/or the at least one sensor.

Example 9 includes the method of example 1, in which the compatibility information maps a given receiver, to one or more transmitters, and in which the compatibility information maps a given transmitter to one or more receivers.

Example 10 includes the method of example 1, in which an eligibility engine accesses an insurance server including information indicative of whether the host-patient has insurance coverage for purchases associated with the continuous analyte monitoring system.

Example 11 includes the method of example 10, in which the eligibility engine queries, via a cache, the insurance server.

Example 12 includes the method of example 11, in which the cache aggregates queries made to the insurance server, and in which the eligibility engine further access the insurance server to determine co-payment information for purchases associated with the continuous analyte monitoring system.

Example 13 includes the method of example 1, in which an eligibility engine controls ordering in a receiver, transmitter, and sensor sequence.

Example 14 includes the method of example 1, in which the receiving, at the server, the first selection comprises receiving the first selection from the host-patient via a remote server or a display device comprising a smartphone and a tablet.

Example 15 includes the method of example 13, in which the receiving, at the server, the second selection comprises receiving the second selection from the host-patient via the remote server or the display device.

Example 16 includes the method of example 13, in which the receiving, at the server, the third selection comprises receiving the third selection from the host-patient via the remote server or the display device.

Example 17 includes the method of example 13, in which the first user interface view, the second user interface view, and the third user interface view are presented at the remote server or the display device to enable selection by the host-patient.

In some embodiments of the present technology (example 18), an apparatus includes at least one processor including at least one memory including program code which when executed by the at least one processor causes operations including: generating a first user interface view displayable on a display of a user device to enable presentation and/or selection, on the display, of text and/or a graphic representative of at least one receiver for a continuous analyte monitoring system; receiving, from the user device, data corresponding to a first selection of the text and/or the graphic representative of a selected receiver among the at least one receiver; determining, using compatibility information, one or more transmitters that are compatible to the selected receiver; generating a second user interface view displayable on the display of the user device to enable presentation and/or selection, on the display, of text and/or a graphic representative of the one or more compatible transmitter; receiving, from the user device, data corresponding to a second selection of a transmitter of the presented and/or selected one or more compatible transmitters; determining, using compatibility information, one or more sensors that are compatible to the selected transmitter; generating a third user interface view displayable on the display of the user device to enable presentation and/or selection, on the display, of text and/or a graphic representative of the one or more compatible sensors; receiving, from the user device, data corresponding to a third selection of a sensor of the presented and/or selected one or more compatible sensors; and initiating a delivery of the selected receiver, the selected transmitter, and/or the selected sensor, in which the apparatus comprises a server.

Example 19 includes the apparatus of example 18, in which the program code which when executed by the at least one processor causes operations further including determining whether a host-patient using the continuous analyte monitoring system has a single account at the server to enable controlled ordering associated with the continuous analyte monitoring system.

Example 20 includes the apparatus of example 19, in which the program code which when executed by the at least one processor causes operations further including determining whether the host-patient is logged into the single account.

Example 21 includes the apparatus of example 20, in which the program code which when executed by the at least one processor causes operations further including determining whether access, by the host-patient accessing the server, includes a token indicative of having the single account at the server, when the patient is not logged in to the single account.

Example 22 includes the apparatus of example 21, in which the program code which when executed by the at least one processor causes operations further including determining whether an email associated with the host-patient is a unique email at the server, when the access does not include the token.

Example 23 includes the apparatus of example 22, in which the program code which when executed by the at least one processor causes operations further including creating the single account, when patient information obtained from the host-patient accessing the server indicates the host-patient does not have the single account.

Example 24 includes the apparatus of example 23, in which the program code which when executed by the at least one processor causes operations further including performing a first check of a database by querying existing accounts for the host-patient's last name, date of birth, and at least a portion of a serial number of a receiver, a transmitter, and/or a sensor associated with the host-patient; and performing a second check of the database by querying the existing accounts for the host-patient's phone number, when the first check fails to find a matching account for the host-patient.

Example 25 includes the apparatus of example 18, in which an eligibility engine at the server determines the at least one receiver, the at least one transmitter, and/or the at least one sensor using the compatibility information comprising: previous purchases related to the at least one receiver. the at least one transmitter, and/or the at least one sensor; identity information for the at least one receiver, the at least one transmitter, and/or the at least one sensor; a software version being implemented at the at least one receiver, the at least one transmitter, and/or the at least one sensor; and/or a model of the at least, one receiver, the at least one transmitter, and/or the at least one sensor.

Example 26 includes the apparatus of example 18, in which the compatibility information maps a given receiver, to one or more transmitters, and in which the compatibility information maps a given transmitter to one or more receivers.

Example 27 includes the apparatus of example 18, in which an eligibility engine accesses an insurance server including information indicative of whether the host-patient has insurance coverage for purchases associated with the continuous analyte monitoring system.

Example 28 includes the apparatus of example 27, in which the eligibility engine queries, via a cache, the insurance server.

Example 29 includes the apparatus of example 28, in which the cache aggregates queries made to the insurance server, and in which the eligibility engine further access the insurance server to determine co-payment information for purchases associated with the continuous analyte monitoring system.

Example 30 includes the apparatus of example 18, in which an eligibility engine controls ordering in a receiver, transmitter, and sensor sequence.

Example 31 includes the apparatus of example 18, in which the receiving, at the server, the first selection comprises receiving the first selection from the host-patient via a remote server or a display device comprising a smartphone and a tablet.

Example 32 includes the apparatus of example 31, in which the receiving, at the server, the second selection comprises receiving the second selection from the host-patient via the remote server or the display device.

Example 33 includes the apparatus of example 31, in which the receiving, at the server, the third selection comprises receiving the third selection from the host-patient via the remote server or the display device.

Example 34 includes the apparatus of example 31, in which the first user interface view, the second user interface view, and the third user interface view are presented at the remote server or the display device to enable selection by the host-patient.

In some embodiments of the present technology (example 35), a non-transitory computer program product includes instructions that, when executed by at least one programmable processor forming part of at least one computing system, cause the at least one programmable processor to perform operations including generating a first user interface view to enable presentation and/or selection of at least one receiver for a continuous analyte monitoring system; receiving, at a server, a first selection of the at least one receiver; determining, by the server using compatibility information, at least one transmitter that is compatible to the selected at least one receiver; generating a second user interface view to enable presentation and/or selection of the at least one transmitter; receiving, at the server, a second selection of the at least one transmitter; determining, by the server using compatibility information, at least one sensor that is compatible to the selected at least one transmitter; generating a third user interface view to enable presentation and/or selection of the at least one sensor; receiving, at the server, a third selection of the at least one sensor; and initiating a delivery of the selected receiver, the selected at least one transmitter, and/or the selected at least one sensor.

In some embodiments of the present technology (example 36), a method for controlling ordering of supplies for a medical device includes determining, at a server, authorization of a user to submit an order for one or more supplies associated with a medical device prescribed to the user; receiving, at the server, information associated with the authorized user, in which the information includes (i) eligible items permitted to be purchased by the user based on one or more of the user's prescription for the medical device, or compatibility of components associated with the medical device, and (ii) pricing of the eligible items associated with the medical device based on an insurance plan of the user; determining, at the server, eligibility of the user to purchase the one or more supplies submitted in the order including analyzing the order and the eligible items; and determining, at the server, payment information for payers associated with purchase of the determined eligible one or more supplies submitted in the order, in which the payment information for payers include a co-payment for the user and a covered payment for a carrier of the insurance plan.

Example 37 includes the method of example 36, in which the determining the authorization of the user includes determining whether the user has a single account on the server.

Example 37 includes the method of example 36, further including receiving, at the server, the order for the one or more supplies associated with the medical device prescribed to the user, in which the medical device includes a sensor, a transmitter to transmit data acquired by the sensor, and a receiver to receive the transmitted data.

Example 37 includes the method of example 38, in which the receiving the order includes: generating a first user interface view to enable presentation and/or selection of at least one receiver component associated with the medical device; receiving, at the server, a first selection of the at least one receiver; determining, at the server, using the information, at least one transmitter that is compatible to the selected at least one receiver; generating a second user interface view to enable presentation and/or selection of the at least one transmitter associated with the medical device; receiving, at the server, a second selection of the at least one transmitter; determining, by the server using compatibility information, at least one sensor that is compatible to the selected at least one transmitter; generating a third user interface view to enable presentation and/or selection of the at least one sensor associated with the medical device; receiving, at the server, a third selection of the at least one sensor; and initiating a delivery of the selected receiver, the selected at least one transmitter, and/or the selected at least one sensor.

Example 37 includes the method of example 36, in which the receiving the information pertaining to the pricing includes receiving at least some of the information from a computer operated by the carrier of the insurance plan.

In some embodiments of the present technology (example 41), an apparatus includes at least one processor including at least one memory including program code which when executed by the at least one processor causes operations including: determining authorization of a user to submit an order for one or more supplies associated with a medical device prescribed to the user; receiving information associated with the authorized user, in which the information includes (i) eligible items permitted to be purchased by the user based on one or more of the user's prescription for the medical device, or compatibility of components associated with the medical device, and (ii) pricing of the items associated with the medical device based on an insurance plan of the user; determining eligibility of the user to purchase the one or more supplies submitted in the order including analyzing the order and the eligible items; and determining payment information for payers associated with purchase of the determined eligible one or more supplies submitted in the order, in which the payment information for payers include a co-payment for the user and a covered payment for a carrier of the insurance plan.

Example 42 includes the apparatus of example 41, in which the determining the authorization of the user includes determining whether the user has a single account on the server.

Example 43 includes the apparatus of example 41, in which the program code which when executed by the at least one processor causes operations further including receiving, at the server, the order for the one or more supplies associated with the medical device prescribed to the user, in which the medical device includes a sensor, a transmitter to transmit data acquired by the sensor, and a receiver to receive the transmitted data.

Example 44 includes the apparatus of example 43, in which the receiving the order includes: generating a first user interface view to enable presentation and/or selection of at least one receiver component associated with the medical device; receiving, at the server, a first selection of the at least one receiver; determining, at the server, using the information, at least one transmitter that is compatible to the selected at least one receiver; generating a second user interface view to enable presentation and/or selection of the at least one transmitter associated with the medical device; receiving, at the server, a second selection of the at least one transmitter; determining, by the server using compatibility information, at least one sensor that is compatible to the selected at least one transmitter; generating a third user interface view to enable presentation and/or selection of the at least one sensor associated with the medical device; receiving, at the server, a third selection of the at least one sensor; and initiating a delivery of the selected receiver, the selected at least one transmitter, and/or the selected at least one sensor.

Example 45 includes the apparatus of example 41, in which the receiving the information pertaining to the pricing includes receiving at least some of the information from a computer operated by the carrier of the insurance plan.

In some embodiments of the present technology (example 46), a method for controlling ordering of supplies for a medical device includes determining, at a server, authorization of a user to submit an order for one or more supplies associated with a medical device prescribed to the user; receiving, at the server, information associated with the authorized user, in which the information includes (i) eligible items permitted to be purchased by the user based on one or more of (a) the user's prescription for the medical device or (b) compatibility of components associated with the medical device, and (ii) pricing of the eligible items associated with the medical device based on an insurance plan of the user; and determining, at the server, eligibility of the user to purchase the one or more supplies submitted in the order based on analysis the information.

Example 47 includes the method of example 46, further including determining, at the server, payment information for payers associated with purchase of the determined eligible one or more supplies submitted in the order, in which the payment information for payers include a co-payment for the user and a covered payment for a carrier of the insurance plan

Example 48 includes the method of example 46, in which the determining the authorization of the user includes determining whether the user has a single account on the server.

Example 49 includes the method of example 48, in which the receiving the information pertaining to the pricing includes receiving at least some of the information from a computer operated by the carrier of the insurance plan.

Example 50 includes the method of example 46, further including receiving, at the server, the order for the one or more supplies associated with the medical device prescribed to the user, in which the medical device is a continuous analyte monitor, and the components of the medical device include a receiver, a transmitter, and at least one sensor.

Example 51 includes the method of example 50, in which the receiving the order includes generating a first user interface view to enable presentation and/or selection of at least one receiver component associated with the medical device; receiving, at the server, a first selection of the at least one receiver; determining, at the server using the information, at least one transmitter that is compatible to the selected at least one receiver; generating a second user interface view to enable presentation and/or selection of the at least one transmitter associated with the medical device; receiving, at the server, a second selection of the at least one transmitter; determining, at the server using information, at least one sensor that is compatible to the selected at least one transmitter; generating a third user interface view to enable presentation and/or selection of the at least one sensor associated with the medical device; and receiving, at the server, a third selection of the at least one sensor.

Example 52 includes the method of example 50, in which the receiving the order includes generating a user interface view to enable presentation and/or selection of one or more receiver components associated with the medical device, one or more transmitter components associated with the medical device, and at least one sensor associated with the medical device, the generating including, prior to presentation of the user interface, determining that the one or more transmitter components are compatible to the one or more receiver components based on the analysis of the information, and that the at least one sensor is compatible to the one or more transmitter components.

Example 53 includes the method of example 50, further including initiating a delivery of the selected receiver, the selected at least one transmitter, and/or the selected at least one sensor.

Example 54 includes the method of example 46, in which the analysis of the information includes analyzing the compatibility of at least one component associated with the medical device among the eligible items with at least one other component associated with the medical device, in which the compatibility of the components is based on one or more of a model of at least some of the components, an identity of at least some of the components, a previous purchase of at least some of the components, or a software version of at least some of the components.

Example 55 includes the method of example 54, in which the medical device is a continuous analyte monitor, and the components of the medical device include a receiver, a transmitter, and at least one sensor, and in which, when the receiver is the user's mobile device including a smartphone, tablet, or smartwatch, the components of the medical device include a software application operable on the receiver.

In some embodiments of the present technology (example 56), a system for controlling ordering of supplies for a medical device includes a controlled ordering software application operable on a user computing device to (i) receive user input indicative of an order for the one or more supplies associated with a medical device prescribed to a patient user, and (ii) cause transmission of the user input by the user computing device; and one or more secure computers including at least one processor and at least one memory including program code which when executed by the at least one processor causes operations of the one or more secure computers, comprising: receiving the user input, determining authorization of the patient user to submit the order for one or more supplies associated with a medical device, processing ordering information associated with the authorized patient user, in which the ordering information includes (i) eligible items permitted to be purchased by the patient user based on one or more of (a) the patient user's prescription for the medical device or (b) compatibility of components associated with the medical device, and (ii) pricing of the eligible items associated with the medical device based on an insurance plan of the patient user, and determining eligibility of the patient user to purchase the one or more supplies submitted in the order based on the processing of the ordering information.

Example 57 includes the system of example 56, in which the program code, which when executed by the at least one processor of the one or more secure computers, causes operations further comprising determining payment information for payers associated with purchase of the determined eligible one or more supplies submitted in the order, in which the payment information for payers include a co-payment for the patient user and a covered payment for a carrier of the insurance plan.

Example 58 includes the system of example 56, in which the medical device is a continuous analyte monitor, and the components of the medical device include a receiver, a transmitter, and at least one sensor.

Example 59 includes the system of example 58, in which the program code, which when executed by the at least one processor of the one or more secure computers, causes operations further comprising: determining that one or more transmitter components associated with the medical device are compatible to the one or more receiver components associated with the medical device based on the processing of the ordering information, and that one or more sensor components associated with the medical device are compatible to the one or more transmitter components, generating a limited user interface to enable presentation and/or selection of the determined compatible one or more receiver components, one or more transmitter components, and/or one or more sensor components, and providing the limited user interface to the user computer device.

Example 60 includes the system of example 56, in which determination of the authorization of the patient user includes determining whether the patient user has a single account on the server.

Example 61 includes the system of example 56, in which the processing of the ordering information includes analyzing the compatibility of at least one component associated with the medical device among the eligible items with at least one other component associated with the medical device, in which the compatibility of the components is based on one or more of a model of at least some of the components, an identity of at least some of the components, a previous purchase of at least some of the components, or a software version of at least some of the components.

In some embodiments of the present technology (example 62), a computing apparatus includes at least one processor including at least one memory including program code which when executed by the at least one processor causes operations comprising: determining, at a server, authorization of a patient user of a medical device to submit an order for one or more supplies associated with the medical device prescribed to the patient user; receiving, at the server, information associated with the authorized patient user, in which the information includes (i) eligible items permitted to be purchased by the patient user based on one or more of (a) the patient user's prescription for the medical device or (b) compatibility of components associated with the medical device, and (ii) pricing of the eligible items associated with the medical device based on an insurance plan of the patient user; and determining, at the server, eligibility of the patient user to purchase the one or more supplies submitted in the order based on analysis the information.

Example 63 includes the apparatus of example 62, in which the program code which when executed by the at least one processor causes operations further comprising receiving, at the server, the order for the one or more supplies associated with the medical device prescribed to the patient user, in which the medical device includes a sensor, a transmitter to transmit data acquired by the sensor, and a receiver to receive the transmitted data; and initiating a delivery of the selected receiver, the selected at least one transmitter, and/or the selected at least one sensor.

Example 64 includes the apparatus of example 63, in which the receiving the order includes generating a first user interface view to enable presentation and/or selection of at least one receiver component associated with the medical device; receiving, at the server, a first selection of the at least one receiver; determining, at the server using the information, at least one transmitter that is compatible to the selected at least one receiver; generating a second user interface view to enable presentation and/or selection of the at least one transmitter associated with the medical device; receiving, at the server, a second selection of the at least one transmitter; determining, at the server using information, at least one sensor that is compatible to the selected at least one transmitter; generating a third user interface view to enable presentation and/or selection of the at least one sensor associated with the medical device; and receiving, at the server, a third selection of the at least one sensor.

Example 65 includes the apparatus of example 63, in which the receiving the order includes generating a user interface view to enable presentation and/or selection of one or more receiver components associated with the medical device, one or more transmitter components associated with the medical device, and at least one sensor associated with the medical device, the generating including, prior to presentation of the user interface, determining that the one or more transmitter components are compatible to the one or more receiver components based on the analysis of the information, and that the at least one sensor is compatible to the one or more transmitter components.

Example 66 includes the apparatus of example 62, in which the program code which when executed by the at least one processor causes operations further comprising determining, at the server, payment information for payers associated with purchase of the determined eligible one or more supplies submitted in the order, in which the payment information for payers include a co-payment for the patient user and a covered payment for a carrier of the insurance plan.

Example 67 includes the apparatus of example 66, in which the receiving the information pertaining to the pricing includes receiving at least some of the information from a computer operated by the carrier of the insurance plan.

Example 68 includes the apparatus of example 62, in which the analysis of the information includes analyzing the compatibility of at least one component associated with the medical device among the eligible items with at least one other component associated with the medical device, in which the compatibility of the components is based on one or more of a model of at least some of the components, an identity of at least some of the components, a previous purchase of at least some of the components, or a software version of at least some of the components.

Various implementations of the subject matter described herein may be realized in digital electronic circuitry, integrated circuitry, specially designed ASICs (application specific integrated circuits), computer hardware, firmware, software, and/or combinations thereof. The circuitry may be affixed to a printed circuit board (PCB), or the like, and may take a variety of forms, as noted. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications, or code) include machine instructions for a programmable processor, and may be implemented in a high-level procedural and/or object-oriented programming language, and/or in assembly/machine language. As used herein, the term “machine-readable medium” refers to any non-transitory computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the subject matter described herein may be implemented on a computer having a display device (e.g., a CRT (cathode ray tube) or LCD (liquid crystal display) monitor) for displaying information to the user and a keyboard and a pointing device (e.g., a mouse or a trackball) by which the user may provide input to the computer. Other kinds of devices may be used to provide for interaction with a user as well; for example, feedback provided to the user may be any form of sensory feedback (e.g., visual feedback, audible feedback, or tactile feedback); and input from the user may be received in any form, including acoustic, speech, or tactile input.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

Although a few variations have been described in detail above, other modifications are possible. For example, while the descriptions of specific implementations of the current subject matter discuss analytic applications, the current subject matter is applicable to other types of software and data services access as well. Moreover, although the above description refers to specific products, other products may be used as well. In addition, the logic flows depicted in the accompanying figures and described herein do not require the particular order shown, or sequential order, to achieve desirable results. Other implementations may be within the scope of the following claims.

For ease of explanation and illustration, in some instances the detailed description describes exemplary systems and methods in terms of a continuous glucose monitoring environment; however it should be understood that the scope of the invention is not limited to that particular environment, and that one skilled in the art will appreciate that the systems and methods described herein can be embodied in various forms. Accordingly any structural and/or functional details disclosed herein are not to be interpreted as limiting the systems and methods, but rather are provided as attributes of a representative embodiment and/or arrangement for teaching one skilled in the art one or more ways to implement the systems and methods, which may be advantageous in other contexts.

For example, and without limitation, described monitoring systems and methods may include sensors that measure the concentration of one or more analytes (for instance glucose, lactate, potassium, pH, cholesterol, isoprene, and/or hemoglobin) and/or other blood or bodily fluid constituents of or relevant to a host and/or another party.

By way of example, and without limitation, monitoring system and method embodiments described herein may include finger-stick blood sampling, blood analyte test strips, non-invasive sensors, wearable monitors (e.g. smart bracelets, smart watches, smart rings, smart necklaces or pendants, workout monitors, fitness monitors, health and/or medical monitors, clip-on monitors, and the like), adhesive sensors, smart textiles and/or clothing incorporating sensors, shoe inserts and/or insoles that include sensors, transdermal (i.e. transcutaneous) sensors, and/or swallowed, inhaled or implantable sensors.

In some embodiments, and without limitation, monitoring systems and methods may comprise other sensors instead of or in additional to the sensors described herein, such as inertial measurement units including accelerometers, gyroscopes, magnetometers and/or barometers; motion, altitude, position, and/or location sensors; biometric sensors; optical sensors including for instance optical heart rate monitors, photoplethysmogram (PPG)/pulse oximeters, fluorescence monitors, and cameras; wearable electrodes; electrocardiogram (EKG or ECG), electroencephalography (EEG), and/or electromyography (EMG) sensors; chemical sensors; flexible sensors for instance for measuring stretch, displacement, pressure, weight, or impact; galvanometric sensors, capacitive sensors, electric field sensors, temperature/thermal sensors, microphones, vibration sensors, ultrasound sensors, piezoelectric/piezoresistive sensors, and/or transducers for measuring information of or relevant to a host and/or another party.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method for controlling ordering of supplies for a medical device, comprising:
determining, at a server, authorization of a user to submit an order for one or more supplies associated with a medical device prescribed to the user;
receiving, at the server, information associated with the authorized user, wherein the information includes (i) eligible items permitted to be purchased by the user based on one or more of (a) the user's prescription for the medical device or (b) compatibility of components associated with the medical device, and (ii) pricing of the eligible items associated with the medical device based on an insurance plan of the user;
determining, at the server, eligibility of the user to purchase the one or more supplies submitted in the order based on analysis the information; and
determining, at the server, payment information for payers associated with purchase of the determined eligible one or more supplies submitted in the order, wherein the payment information for payers include a co-payment for the user and a covered payment for a carrier of the insurance plan.
